(19) United States
(12) Patent Application Publication
(10) Pub. No.: US 2012/0290065 A1
(43) Pub. Date: Nov. 15, 2012

(54) Pre-Positioned Anastomosis Device and Related Methods of Use

(75) Inventors: Jianmin Li, Lexington, MA (US); Ty Fairneny, Hopkinton, MA (US); Steven A. Olivieri, Shrewsbury, MA (US)

(73) Assignee: Boston Scientific Scimed Inc.

(21) Appl. No.: 13/465,252

(22) Filed: May 7, 2012

Related U.S. Application Data
(60) Provisional application No. 61/485,353, filed on May 12, 2011.

(51) Int. Cl. A61F 2/84; A61F 2/82

(52) U.S. Cl. 623/1.11; 623/1.36

(57) ABSTRACT

Embodiments of a medical device and related methods of use are provided in the disclosure. The medical device includes an elongate member having a first end defining a first opening, a second end defining a second opening, and a tapered lumen extending between the first and second openings. The diameter of the first end may be smaller than that of the second end. The medical device also may include a securing mechanism protruding from one of the first or second ends to penetrate tissue.

### CROSS-REFERENCE TO RELATED APPLICATIONS

This patent application claims the benefits of priority under 35 U.S.C. §§119-120 to U.S. Provisional Application No. 61/485,353, filed May 12, 2011, entitled PRE-POSITIONED ANASTOMOSIS DEVICE AND RELATED METHODS OF USE, the entirety of which is incorporated herein by reference.

### FIELD OF THE INVENTION

Embodiments of this disclosure relate generally to medical devices and procedures, and more particularly to medical devices and procedures for anastomosis.

### BACKGROUND OF THE INVENTION

Bladder obstruction, arising from enlargement of the prostate gland in males, is a commonly occurring disorder in urology. The prostate gland lies under the bladder and surrounds the passageway known as the prostatic urethra, which transfers fluids from the bladder to the urethral sphincter and ultimately outside the body through the urethra. An enlarged prostate gland constricts the prostatic urethra, causing a condition known as benign prostatic hyperplasia (“BPH”). BPH in turn presents a variety of obstructive symptoms including urinary hesitancy, decreased size and force of the urinary stream, and, in extreme cases, cause urinary retention. That condition is highly dangerous, as it can lead to renal failure.

One treatment for bladder obstruction is radical prostatectomy. That procedure involves surgically removing the prostate gland after severing it from the bladder neck and the urethra. Anastomosis is then undertaken to re-attach the urethral stump to the bladder neck. That procedure is often complicated by the tendency of the urethral stump to retract into adjacent tissue. When that happens, considerable time and effort can be required to re-expose the urethral stump and complete the anastomosis. Moreover, the urethral stump may be hidden beneath the pubic bone, requiring the surgeon to work at a difficult angle and in uncomfortable positions.

Conventionally, a surgeon may execute delicate suturing operations with fine needles to reconnect these anatomical bodies. Deployment of sutures to connect the severed tissues, however, can be a difficult and often technique-challenging task. Moreover, inappropriate suturing can result in complications such as leakage, difficulty in healing or failure to heal, incontinence, or impotence. While there have been some attempts to provide improved devices and methods for anastomosis in radical prostatectomy, these attempts have not been entirely successful

Thus, a device that ensures effective anastomosis during radical prostatectomy is desirable.

### SUMMARY OF THE INVENTION

Embodiments of the disclosure provide a medical device and its related methods of use. In some embodiments, the medical device may be used for maintaining the patency of a prostatic urethra during removal of a patient's prostate.

In accordance with an aspect of the present disclosure, a medical device includes a self-expandable elongate member having a first end defining a first opening, a second end defining a second opening, and a tapered lumen extending between the first and second openings. In addition, a diameter of the first opening may be smaller than a diameter of the second opening. An outer surface of the elongate member may include at least one score line configured to facilitate separation of the first end from the second end. Further, the medical device may also include a securing mechanism protruding from the first end or the second. The securing mechanism may be configured to penetrate body tissues to anchor the medical device. The elongate member or the securing mechanism may be biodegradable.

In various embodiments, the medical device may include one or more of the following additional features: the securing mechanism may be disposed at the first and second ends; the entire medical device may be biodegradable; the securing mechanism may include barbs or spikes configured to penetrate through the body tissues; the securing mechanism may include an opening configured to receive a sutures; the securing mechanism may include an expandable member, such as a balloon; different portions of the medical device may biodegrade at different rates; a portion of the medical device may include a coating such as a coating of a lubricous material or an anti-biotic agent; the medical device may include one of radioopaque or sonoreflective markings assisting in appropriate positioning of the medical device within the body; the at least one score line may include two score lines; and one of the two score lines may be disposed closer to the first end than the other of the two score lines.

According to another embodiment, a medical device delivery system may include an elongated tubular delivery member and an expandable, bio-degradable medical device disposed in a lumen of the delivery member. The medical device may include a first end having a first opening, a second end having a second opening, and a lumen extending between the first and second openings. A securing mechanism, configured to penetrate body tissues, may be disposed at one of the first and second end of the medical device, and a score line may be disposed on an exterior surface of the medical device at a location between the first and second openings.

In various embodiments, the medical device may include one or more of the following additional features: the first opening may be larger than the second opening; the securing mechanism may include one of spikes and barbs; the securing mechanism may include a hole disposed in an exterior surface of the medical device and a suture extending therethrough; and the securing mechanism may be disposed at both the first and second ends.

A further aspect of the present disclosure may include a method of positioning a urethral anastomosis. The method may include advancing a catheter within a bladder and/or urethra of a patient. The catheter may include a medical device disposed within a lumen of the catheter. The medical device may include a first end having a first opening, a second end having a second opening, and a lumen extending between the first and second openings. The first opening may be larger than the second opening. The medical device also may include a plurality of projections disposed at one of the first or second ends to, among other things, anchor the device to body tissues. The method may further include advancing the medical device out of the lumen of the catheter. The advancing step may include expansion of the medical device once the catheter releases the medical device. Subsequently, the projections of the medical device may engage the urethra and/or the bladder.

In various embodiments, the medical device may include one or more of the following additional features: the plurality of projections may be disposed at the first and second ends; the plurality of projections may be configured to pierce tissue; the plurality of projections may include one of barbs and spikes; and the lumen may taper from the first opening to the second opening.

In another aspect, the present disclosure includes a method of positioning an anastomosis proximate a prostate. The method may include the step of advancing a catheter including a bio-degradable medical device to a position within one or both of a urethra and a bladder of a patient. The medical device may include a first end having a first opening, a second end having a second opening, and a lumen extending between the first and second openings. In addition, the medical device may include a plurality of securing mechanisms disposed at one of the first and second ends and a plurality of score lines defining a removable portion of the medical device therebetween. The method may also include advancing the medical device out of a lumen of the catheter to permit expansion of the medical device and positioning the medical device such that the removable portion is proximate the prostate. The method may further include engaging the plurality of securing mechanisms with a portion of one or both of the urethra and bladder.

In some embodiments, the method may further include removing the removable portion of the medical device and the prostate, and re-connecting severed portions of the medical device.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate embodiments of the disclosure and, together with the description, serve to explain the principles of the disclosure.

FIG. 1 is a schematic view of the male urinary system.

FIG. 2A is a schematic view of a medical device in a deployed state according to an embodiment of the present disclosure.

FIG. 2B is a schematic view of the medical device (shown in FIG. 2A) before insertion into the urinary system according to an embodiment of the present disclosure.

FIG. 3 is a perspective view of the medical device, shown in FIG. 2A, having barbs as the securing mechanism.

FIG. 4 is a schematic view of the medical device of FIG. 2A, having barbs disposed only at the distal end.

FIG. 5 is a schematic view of a further embodiment of a medical device of the present disclosure, having hooks disposed at its ends.

FIG. 6 is a schematic view of the medical device, shown in FIG. 2A, having an expandable balloon connected to one end acting as a securing mechanism.

FIGS. 7A, 7B, 7C, and 7D are schematic representations of, respectively, the medical device of FIG. 2A being placed within a patient's prostatic urethra using a catheter; the medical device being deployed from the catheter; the patient's prostate being removed; and the healing process underway.

### DESCRIPTION OF THE EMBODIMENTS

Reference will now be made in detail to embodiments of the invention, an example of which is illustrated in the accompanying drawings. Wherever possible, the same reference numbers will be used throughout the drawings to refer to the same or like parts.

### Overview

The present disclosure provides medical devices, such as medical stents, configured to be positioned, or pre-positioned, within a patient's body. In particular, an embodiment of the disclosed medical device may be pre-positioned within the urethra to facilitate drainage of urine from the patient's bladder during and/or after radical prostatectomy. In addition, pre-positioned medical devices assist in healing after prostate removal. Although exemplary embodiments of the present disclosure are described with reference to radical prostatectomy, it will be appreciated that aspects of the present disclosure have wide application and may be suitable for use in other body locations. For example, the medical device may be placed within a bile duct for use as a biliary stent.

Generally, the medical device according to the present disclosure includes a hollow elongate structure having a first end referred to as a distal end, a second end referred to as a proximal end, and a longitudinally extending lumen between the two ends. The term “distal” refers to the end of the medical device farthest away from a medical professional when placing the medical device in a patient. By contrast, “proximal” refers to the end of the medical device closest to the medical professional when placing the medical device in the patient. The radius of the distal end may be substantially greater than the proximal end.

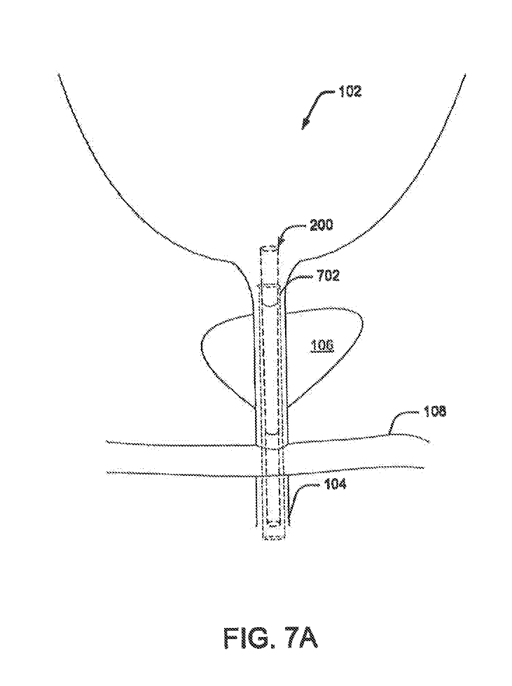

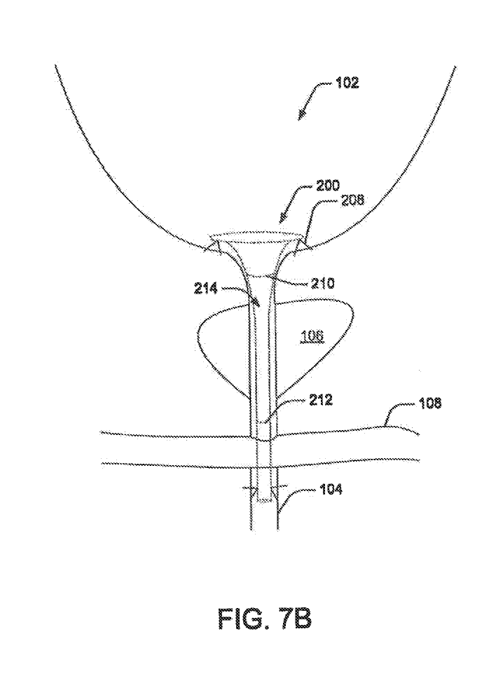

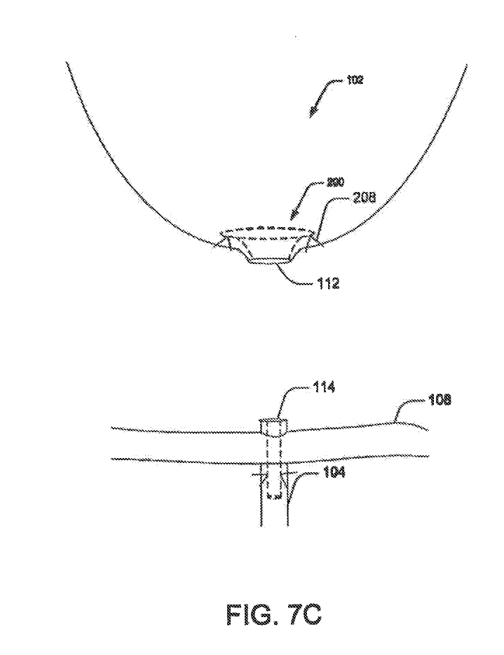

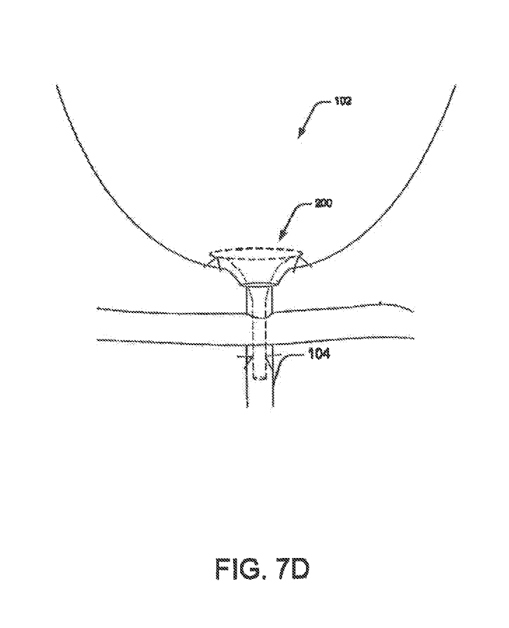

The medical device may include a self-expanding structure that appropriately positions itself within the bladder and extends along the urethra. In some embodiments, however, the disclosed medical device may be configured for expansion by any suitable means known in the art, including, for example, expansion by a balloon. Further, the medical device may include securing mechanisms disposed at the distal or proximal ends to mitigate migration after placement. The design of the medical device and the incorporated securing device provide an effective and comfortable way to anchor the medical device within the body. Once the medical device is appropriately anchored within the bladder, radical prostatectomy, or any other suitable procedure, if necessary may be performed. For example, the surgeon may remove the prostate along with a separable section of the medical device that is surrounded by the prostate. Subsequently, the severed tissues of the bladder neck and urethral stump may be reconnected with the help of the medical device, as illustrated in FIGS. 7C & 7D. The united medical device enables urine flow from the bladder to urethra during healing. Those skilled in the art will understand that pre-positioning the medical device within the body during, e.g., radical prostatectomy inhibits the urethral stump from retracting into adjacent tissue, and make it easy for physicians to locate the stump and bladder neck for urethra reconnection. In addition, embodiments of the disclosed devices may be made of a bio-resorbable material, which may enable resorbtion of the medical device material once anastomosis and healing is complete, avoiding of the need to remove the medical device after healing.

### Exemplary Embodiments

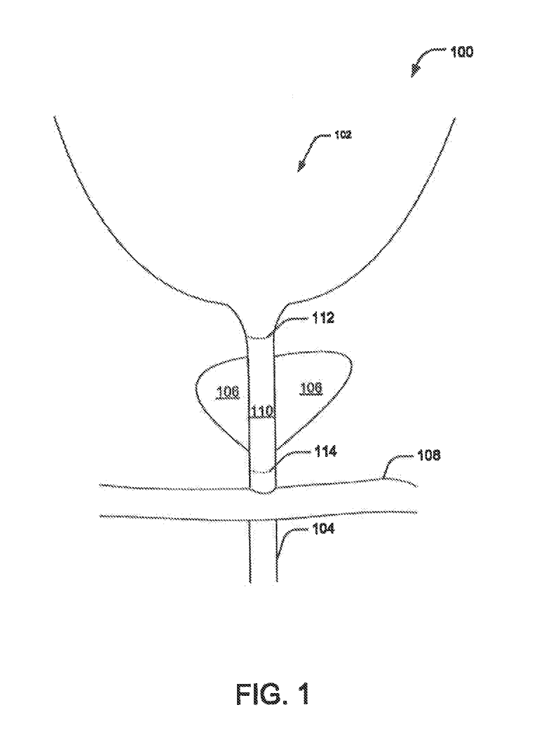

FIG. 1 illustrates a typical male urinary system 100 that may be afflicted with benign prostatic hyperplasia (BPH). The system 100 includes a bladder 102, a urethra 104, a prostate 106, and a perineal floor 108. The prostate 106 is a male reproductive organ that surrounds a section of the urethra 104 generally known as the prostatic urethra 110. It should be understood that BPH may enlarge the prostate 106 and result in constriction and obstruction of the male urinary system 100. One potential treatment for patients suffering from BPH includes undergoing radical prostatectomy or other similar procedures that remove the prostate 106 and the prostatic urethra 110. The surgical procedure may leave the bladder 102 with the severed tissue surface of the bladder neck 112 opposing the severed tissue surface of the urethral stump 114 extending from the perineal floor 108.

To maintain an open passageway through the prostatic urethra 110 during the healing period, the embodiments of the present disclosure employ a medical anastomosis device similar to a temporary prostate stent. Although the present disclosure discusses the medical device in relation to radical prostatectomy, it will be understood that the device is applicable for use in any anastomotic procedure where the end of a conduit is to be sutured or secured to a hollow body organ. Furthermore, it will be readily apparent to those of ordinary skill in the art that embodiments of the present disclosure may be employed in other body lumens, including, for example, blood vessels, bile ducts, and the gastrointestinal tract.

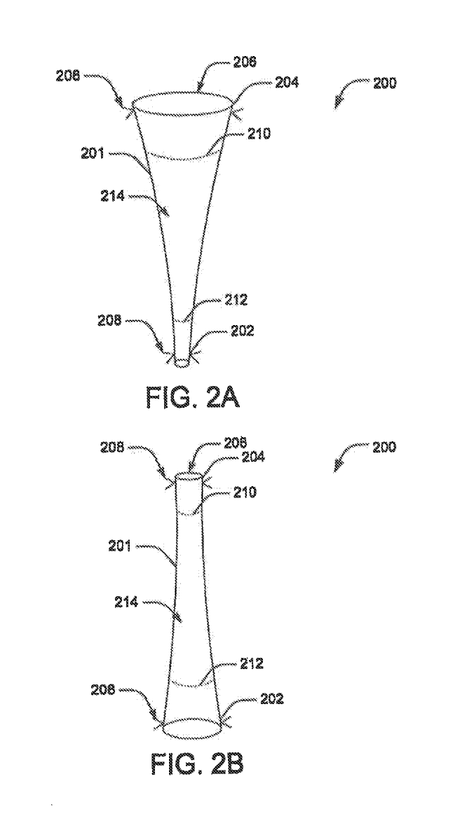

FIG. 2A illustrates an exemplary medical device 200 that can be implanted in a patient's body. The medical device 200 may include an elongate member 201 having a proximal end 202, a distal end 204, and a hollow, tapered lumen 206 extending between the proximal end 202 and distal end 204. The distal end 204 may have a wider diameter than that of the proximal end 202, to facilitate anchoring on the bladder neck 112. To anchor the medical device 200 in a desired position within the patient's body, a security/retention mechanism 208, protruding from one or both of the proximal end 202 and distal end 204, may engage tissue to hold the device 200 in place. Further, score lines 210, 212 may define a separable section 214 on the elongate member, between the proximal end 202 and distal end 204.

The medical device 200 may be made of any suitable material that is compatible with living tissue or a living system, non-toxic or non-injurious, and does not cause immunological reaction or rejection. Such materials may include, including, for example, polymers, nitinol, ePTFE, fabric, and suitable nickel and titanium alloys. In some embodiments, device 200 may be made of a biodegradable or bioresorbable material. For example, device 200 may decompose or degenerate into a water-soluble substance, which dissolves or erodes over time upon exposure to a body fluid such as urine. In other implementations, the medical device material may not be soluble, but it will degrade into sufficiently fine particles that can be suspended in the body fluid and transported away from the implantation site without clogging the flow of the body fluid. As a result, once the anastomosis is complete and sufficient healing has occurred, the medical device may be eliminated from the body by excretion or metabolized by the body.

In one embodiment of the present disclosure, the medical device material may comprise a resorption profile of varying resorption rates. The resorption profile can be a function of time such as two or more differing sequential rates (e.g., slow initial resorption and fast late-stage resorption, or vice versa). The resorption profile can also be a function of a physical dimension of the implant (e.g., slow resorption of one portion and fast resorption of another portion). For example, the proximal end 202 may resorb faster than the distal end 204.

To inhibit migration of the medical device 200 once placed, the medical device 200 may include a suitable securing mechanism 208. Securing mechanism 208 may be geometrical features protruding from the elongate member 201 to engage body tissues. Those skilled in the art will understand that a variety of known securing mechanisms, such as hooks, barbs, or spikes and diameter differences between the medical device and the bladder neck/urethra, may be employed to mitigate migration of the medical device 200 from a deployed position. In some embodiments, securing mechanism 208 may be selectively deployed and/or withdrawn.

Medical device 200 may be pre-folded and self-expandable, as set out more fully in connection with FIGS. 7A-7D. The device 200 may be formed of smooth material, sufficiently flexible to conform to the bodily location in which it is employed, yet sufficiently rigid to maintain the patency of lumen 206. In the configuration shown in FIG. 2A, the radius of distal end 204 may be large enough to anchor it within the bladder neck 102; the elongate member 201 may be long enough to extend a desired length along the prostatic urethra 110, and the proximal end may be sized to fit within the urethra 104. Further, the length of the device 200 may vary depending on the size of the patient's prostatic urethra 110, which may vary in length from about 1 cm to about 8 cm. To determine the length of the patient's prostatic urethra 110, a conventional measuring catheter can be employed. The diameter of the lumen 206 can be varied to fit given patients. By varying the diameter and length, the medical device 200 may be tailored to the individual needs of particular patients.

In addition, a portion or the entire medical device 200 may include a coating. In one embodiment of the present disclosure, the medical device 200 may be coated with anti-biotics or anti-microbial to inhibit bacterial growth on the surface of the medical device 200. The anti-biotic coating may contain an inorganic antibiotic agent disposed in a polymeric matrix, which adheres the antibiotic agent to the medical device surface. Further, a drug releasing coating may also be applied to the outer surface of the medical device 200, assisting in healing. In another embodiment of the present disclosure, the medical device 200 may be coated with a lubricious coating to facilitate insertion in the bladder 102 and urethra 104. The lubricious coating on the medical device 200 may reduce the frictional forces between device 200 and the tissue it may contact.

In the embodiment shown in FIG. 2A, elongate member 201 may be tapered, with distal end 204 having a diameter larger than that of proximal end 202. Those in the art will understand that member 201 may be configured or folded into a taper as shown. The illustrated embodiment depicts the deployed shape of the elongate member 201 with distal end 204 having a greater diameter than the proximal end 202. Deploying the device 200 with this configuration may impose hindrances to the surrounding tissues. As a result, the medical device 200 is manufactured with either a uniform diameter (without a taper at all) or with an opposite tapered configuration (where the proximal end 202 has a larger diameter than the distal end 204). FIG. 2B depicts this configuration of the medical device 200 utilized for insertion into a urinary system. The illustrated opposite tapered configuration assist in inserting the device 200 transurethrally. During insertion, the diameter of the distal end 204 should be equal to or lesser than that of the proximal end 202. Once deployed at the desired location, the device 200 may expand to exhibit the configuration depicted in FIG. 2A. Moreover, the elongate member 201 need not exhibit a round, or even uniform, cross-section, but rather its form can be altered to meet existing needs.

Elongate member 201 may be provided in a variety of forms, based on the needs of particular applications. In some embodiments, elongate member 201 may include a stent or stent-like configuration. For example, member 201 may include a scaffolding formed from a plurality wires crossing one another so as to create openings or cells throughout the scaffolding. Where such scaffolding is present, it may be coated or covered to reduce friction, or to cover openings or cells, as generally known in the art. In other embodiments, elongate member 201 may be configured as a coil, or it may be formed as a unitary, one piece construction. Those of ordinary skill in the art will readily recognize that such embodiments may also include any suitable coating or covering. Further, the outermost surfaces of member 201 may be provided with geometric features suitable for facilitating retention of member 201 within a patient's body. Such geometric features may include a range of structures known in the art, such as surface roughening.

It may be further desirable to configure either the distal end 204 or the proximal end 202 to avoid trauma or irritation to bodily tissues during insertion or deployment of medical device 200. Those in the art will understand how best to effect such configuration, through measures such as beveling or rounding, or other measures known in the art. It should be apparent that any geometry of the medical device may be employed, so long as fluid flow is not hindered.

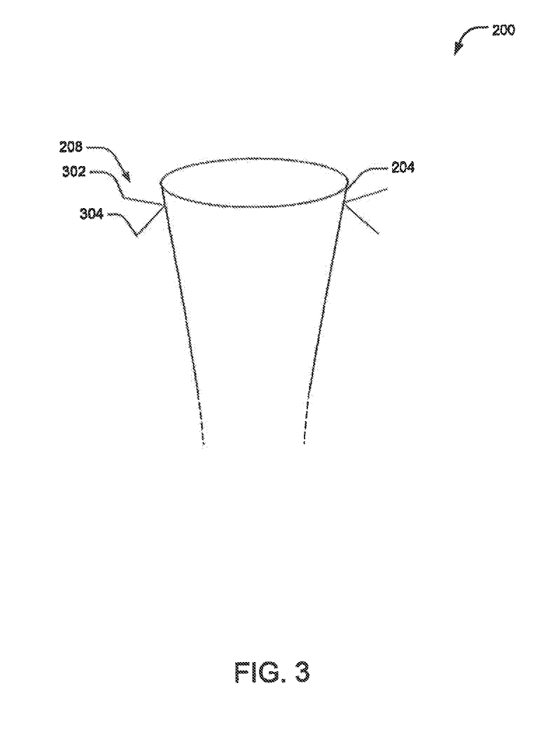

FIG. 3 illustrates a perspective view of the medical device 200 depicting the set of barbs 208 positioned at the distal end 204. Although the depicted embodiment illustrates two sets of barbs 208 positioned diametrically opposite to one another, those of ordinary skill in the art will recognize that distal end 204 may include any suitable number of barbs 208, which may be disposed around distal end 204 at any suitable interval. For example, distal end 204 may include four sets of barbs 208 disposed at 90 degree intervals about end 204. Each barb 208 may include a pair of strut ends 302, 304 protruding outward from the outer surface of the medical device 200 in, e.g., substantially diverging directions. The strut ends 302, 304 may engage body tissue for the purpose of anchoring the medical device 200.

In some embodiments, medical device 200 may include barbs 208 at both the proximal and distal ends 202, 204. In such embodiments, barbs 208 may serve to prevent movement of the medical device 200 in both the proximal and distal directions. That is, barbs 208 may prevent movement of the medical device 200 in the bladder 102 and prevent movement of the medical device 200 down through the urethra 104. Barbs 208 may also be made of a bioresorbable/biodegradable material that degrades over time. In such embodiments, a first set of barbs 208 may be configured to degrade faster than a second set of barbs 208. For example, barbs 208 located at proximal end 202 may be configured to degrade before barbs 208 located at a distal end 204.

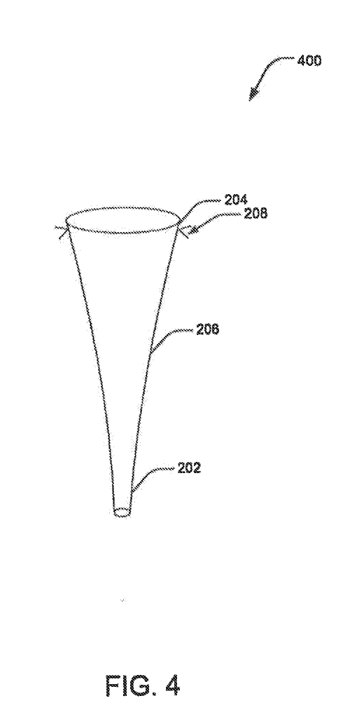

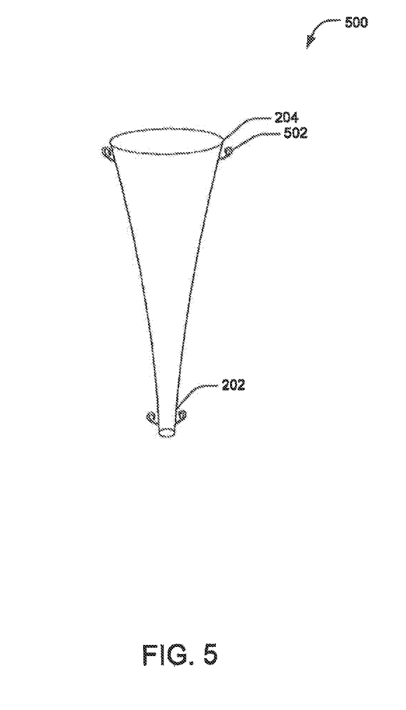

FIGS. 4-6 depict alternate embodiments of the securing or retention mechanism. FIG. 4 illustrates barbs 208 circumferentially positioned only at the distal end 204. It should be understood that the barbs 208 disposed only at the distal end 204 may be enough to hold the medical device 200 in place. In addition, the barbs 208 may protrude not only from the proximal or distal ends 202, 204, but along the length of the tapered lumen 206. In another implementation, the medical device 200 may include openings such as holes disposed at the proximal and/or distal ends 202, 204 to receive a surgical suture or thread. In such embodiments, the device may be sutured or otherwise sewn to selected body tissue.

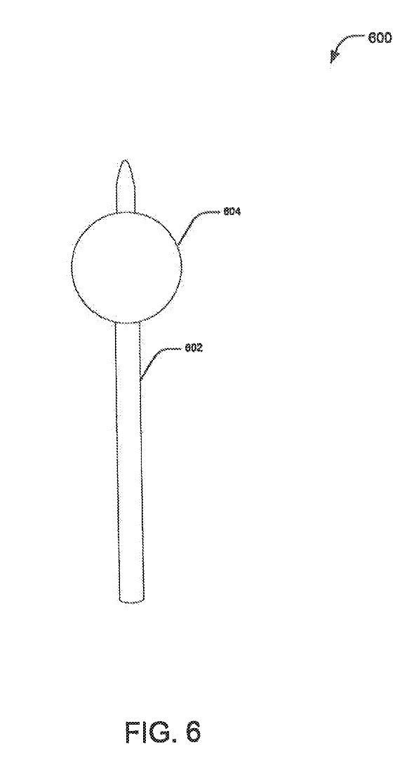

In another embodiment, FIG. 5 depicts hooks 502 as the securing mechanism. As shown, hooks 502 may be positioned at the proximal end 202 and distal end 204. However, those of ordinary skill in the art will recognize that hooks 502 may be positioned at any suitable location, including, e.g., only at one of the proximal and distal ends 202, 204, or, alternatively, along the entire length of medical device 200. Hooks 502 may include any configuration suitable for anchoring device 200 to a patient's tissue. As shown, the hooks 502 may be J-shaped structures or mushroom-shaped heads imposing minimum friction to the body tissues during medical device insertion. Stated differently, hooks 502 may be oriented and/or configured to facilitate insertion of device 200, but inhibit migration of device 200 once it has been deployed.

In yet a further embodiment, an expandable structure may be deployed near the distal end 204 to anchor the medical device 200. A number of such structures, designed as either self-expanding or expanding under control of the operator, are known to those of skill in the art. FIG. 6 illustrates such a device, in the form of an expandable balloon 604 deployed at the end of a cylindrical lumen 602. The balloon 604 may be delivered in a collapsed state, and once inside the bladder 102, the balloon 604 may expand to anchor the device 200 in place. Expandable balloons are well-known in the art, in versions that mechanically expand upon the release of restraint or expand through the injection of water or saline. The selection and employment of such balloons are well known to those in the art.

FIGS. 7A-D illustrate embodiments of the present disclosure including a method of using the medical device 200 to facilitate removal of the prostate 106 from a patient's body. In such embodiments, the medical device 200 may be used to assist in the creation of anastomosis between two severed structures. Those skilled in the art will understand that the present disclosure may be implemented for anastomosis during any surgical procedure, including the removal of any unwanted object from the bladder, urethra, kidney, gastrointestinal tract, or other body structure.

The medical device 200 may be positioned within the bladder 102 so that a proximal portion of the medical device may extend along the urethra 104, as shown in FIG. 7A. A catheter 702 may be employed to facilitate insertion of the medical device 200 to the desired location. As shown, the catheter 702 may be an elongate tube having one or more channels (not shown) that may contain at least a portion of the medical device 200. The catheter 702 containing the medical device 200 may be advanced into the bladder transurethrally. The catheter 702 may be inserted into the urinary system through a natural opening or an incision created in the urethra. The catheter 702 advances the urethra until the distal end of catheter 702 reaches bladder 102. The catheter 702 may include multiple channels that may assist is delivering the medical device, visualizing the delivery location, delivering instruments, and/or irrigation. Those skilled in the art will understand that catheter 702 may also have one or more mechanisms that facilitate steering the distal end of the catheter 702, to aid in appropriate medical device delivery.

Once the catheter 702 reaches the bladder 102 (as shown in FIG. 7A), the medical device 200 may be delivered from a distal end opening of the catheter 702. Once advanced out of the catheter 702, medical device 200 may expand to conform to the urethral and bladder contours. In an alternative embodiment, a suitable expansion mechanism (not shown) may be used to facilitate expansion of medical device 200 after it is delivered to a desired position. Expansion of medical device 200 may cause the securing devices described above, such as barbs, hooks, or spikes, to make contact with and engage nearby tissue, thereby securing the device in position. Subsequently, the catheter 702 may be retracted from the body. Alternatively, additional tools may be advanced to the site of the medical device through catheter 702.

In one embodiment of the present disclosure, the catheter 702 may include mechanisms configured to enable retrieval or repositioning of the medical device 200, in case of inappropriate deployment. For example, the catheter 702 may retrieve a partially deployed medical device 200 into the catheter 702 for repositioning to an optimal location. Insertion, repositioning, and retraction may damage body tissue due to friction between an outer surface of the catheter 702 and surrounding tissues. To reduce this damage, the outer surface of the catheter 702 may be coated with a lubricous coating.

Furthermore, radiopaque or sonoreflective markings (not shown) can be added to the medical device 200 to facilitate detecting the position and orientation of medical device 200 within the patient's body. A surgeon, with the aid of suitable imaging equipment, can view these markings to enable optimal positioning of the medical device 200 and to avoid potential damage to sensitive tissues.

After successful placement of medical device 200 within the patient's body is achieved, additional therapeutic procedures can be performed. For example, in one embodiment, radical prostatectomy can be performed to remove the prostate 106. To this end, the surgeon detaches the prostate 106 along with the separable section 214 of medical device 200 (shown in FIG. 7B). The separable section 214 may be defined by score lines 210, 212 between the proximal end 202 and distal end 204. Generally, the method of the present disclosure includes a step of performing a radical prostatectomy by known techniques such as a retropubic technique, a laparoscopic technique, or a transperineal technique. During radical prostatectomy, the separable section 214 is partially or completely detached from the medical device 200 to remove the prostate 106. This may result in removing the separable section 214 along with the prostate 106 leaving severed tissue surfaces of the bladder neck 112 and urethral stump 114, respectively, as shown in FIG. 7C. Some embodiments of the medical device 200 may not include specific score lines to define the separable section 214, and the surgeon may herself define a separable section to remove the prostate 106. Of course, those of ordinary skill in the art will readily recognize that separable section 214 may be defined or otherwise indicated by any suitable manner known in the art.

In another implementation, the separation of the prostate 106 may require only partial detachment of the medical device 200, and once the prostate 106 is removed, the partially detached medical device 200 may be reconnected to provide a urine drainage path. Suitable methods for reconnection include suturing, attaching a piece of the medical device material, adding known sealants, or other suitable techniques known to the art.

Subsequently, the severed urethral stump tissue may be positioned against the severed surface of the bladder neck to allow healing and reconnection of the two severed tissues, as shown in FIG. 7D. The medical device 200 may facilitate holding the severed tissues in contact with each other during the healing period. Alternatively, the severed surfaces of the medical device may be sutured, sewn, or otherwise reconnected through known reconnection mechanisms. When detached medical device portions proximate to the bladder and the urethra contact each other, the medical device 200 reconnects to form a continuous urine flow path.

The medical device 200 may remain within the body for a predetermined time, to allow for sufficient healing between severed tissue portions. If a bioresorbable embodiment is employed, then the medical device 200 will degrade or resorb over time, which may eliminate the need for a second procedure to remove the medical device 200. The resorption rate of the medical device 200 may vary based on the time required to heal the severed tissues. In one embodiment, the medical device 200 may be configured to begin degrading after, e.g., 8 days.

Embodiments of the disclosure may be used in any medical or non-medical procedure, including any medical procedure where removal of an object from within a body lumen is desired. In addition, at least certain aspects of the aforementioned embodiments may be combined with other aspects of the embodiments, or removed, without departing from the scope of the invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical device comprising:
a self-expandable elongate member having a first end defining a first opening, a second end defining a second opening, and a tapered lumen extending between the first and second openings, a diameter of the first opening being smaller than a diameter of the second opening, and an outer surface of the elongate member including at least one score line configured to facilitate separation of the first end from the second end; and
a securing mechanism configured to protrude from the first end or the second end to penetrate tissue,
wherein at least one of the elongate member and the securing mechanism is biodegradable.

2. The medical device of claim 1, wherein the securing mechanism is disposed at the first and second ends.

3. The medical device of claim 1, wherein the entire medical device is bio-degradable.

4. The medical device of claim 1, wherein the securing mechanism includes one of a barb and a spike.

5. The medical device of claim 1, wherein the securing mechanism includes an opening configured to receive a suture.

6. The medical device of claim 1, wherein the securing mechanism includes an expandable member.

7. The medical device of claim 6, wherein the expandable member includes a balloon.

8. The medical device of claim 1, wherein a first portion of the medical device is configured to biodegrade faster than a second portion of the medical device.

9. The medical device of claim 1, wherein at least one score line includes two score lines.

10. The medical device of claim 9, wherein one of the two score lines is disposed closer to the first end than the other of the two score lines.

11. A medical device delivery system, comprising:
an elongated tubular delivery member; and
an expandable, biodegradable medical device disposed in a lumen of the delivery member, the medical device comprising:
a first end having a first opening, a second end having a second opening, and a lumen extending between the first and second openings;
a securing mechanism disposed at one of the first and second ends, wherein the securing mechanism is configured to penetrate tissue; and
at least one score line disposed on an exterior surface of the medical device at a location between the first and second openings.

12. The medical device of claim 11, wherein the securing mechanism includes one of spikes and barbs.

13. The medical device of claim 11, wherein the first opening is larger than the second opening.

14. The medical device of claim 11, wherein the securing mechanism includes a hole disposed in an exterior surface of the medical device and a suture extending therethrough.

15. The medical device of claim 11, wherein the securing mechanism is disposed at the first and second ends.

16. A method of positioning a urethral anastomosis, the method comprising:
advancing a catheter including a biodegradable medical device to a position within one or both of a urethra and a bladder of a patient, wherein the medical device comprises:
a first end having a first opening, a second end having a second opening, and a lumen extending between the first and second openings, wherein the first opening is larger than the second opening; and
a plurality of projections disposed at one of the first and second ends;
advancing the medical device out of a lumen of the catheter for permitting expansion of the medical device; and
engaging the plurality of projections with a portion of one or both of the urethra and bladder.

17. The method of claim 16, wherein the plurality of projections are disposed at the first and second ends.

18. The method of claim 16, wherein the plurality of projections are configured to pierce tissue.

19. The method of claim 16, wherein the plurality of projections include one of barbs and spikes.

20. The method of claim 16, wherein the lumen tapers from the first opening to the second opening.

21. A method of positioning an anastomosis proximate a prostate, the method comprising:
advancing a catheter including a bio-degradable medical device to a position within one or both of a urethra and a bladder of a patient, wherein the medical device comprises:
a first end having a first opening, a second end having a second opening, and a lumen extending between the first and second openings; and
a plurality of securing mechanisms disposed at one of the first and second ends; and
a plurality of score lines defining a removable portion of the medical device therebetween;
advancing the medical device out of a lumen of the catheter to permit expansion of the medical device;
positioning the medical device such that the removable portion is proximate the prostate; and
engaging the plurality of securing mechanisms with a portion of one or both of the urethra and bladder.

22. The method of claim 21, wherein the method further includes removing the removable portion of the medical device and the prostate, and re-connecting severed portions of the medical device.
